(19) United States
(12) Patent Application Publication
(10) Pub. No.: US 2026/0165584 A1
(43) Pub. Date: Jun. 18, 2026

(54) A Probe for Measuring Spectra, and a System and Method Thereof

(71) Applicant: Zhiwei Huang

(72) Inventor: Zhiwei Huang

(21) Appl. No.: 18/707,815

(22) PCT Filed: Nov. 4, 2022
(86) PCT No.: PCT/SG22/50804

§ 371 Date: May 6, 2024

(51) Int. Cl. A61B 5/00; G01J 3/44

(52) U.S. Cl. CPC A61B 5/0075; A61B 5/0084; A61B 5/7203; G01J 3/44; A61B 2562/0238

(57) ABSTRACT

The invention provides a probe (1) for measuring spectra of a target (21) within a bio-matter (2), comprising an optical structure (11) having a lens portion (111), and a communication line (12) conjoined to the optical structure (11). The optical structure (11) of the probe (1) has its exposed end tapered for forming the lens portion (111) that converges light incoming from the communication line (12) towards the target (21), and converges scattered light incoming from the target (21) towards the communication line (12). A system (3) that incorporates the probe (1), and a method of using the system (3), are further provided.

### RELATED APPLICATION

The present invention claims priority to Singapore patent application Ser. No. 10202112278Q filed on 5 Nov. 2021, the disclosure of which is incorporated in its entirety.

### FIELD OF INVENTION

The invention relates to instruments used for spectra measurements of bio-matter. More particularly, the invention relates to a probe for Raman spectroscopy for probing deep tissues, a system that incorporates this probe, and its method of using this system thereof.

### BACKGROUND OF THE INVENTION

Near-infrared (NIR) Raman spectroscopy refers to a vibrational spectroscopy technique where bio-molecular information of a bio-matter is probed using a probe, especially for label-free characterization and diagnosis of cells and tissue.

Recent developments in Raman probes have enabled in vivo tissue Raman spectroscopy measurements in internal organs (e.g., oesophagus, stomach, colon, and lung etc) under endoscopic guidance. A needle-like Raman probe design has allowed the in vivo tissue Raman measurement through a biopsy needle.

Current needle-like Raman probes designed are usually bulky, having outer diameters ranging from 0.7 mm to 2 mm. They are usually made using either or both multiple fibres and on-tip focusing optics. However, their outer diameter size hampers them from passing through most fine needle aspiration biopsy (FNAB) channels used for deep tissue Raman measurements, which typically have an inner diameter of not more than 0.5 mm. Therefore, it is highly desirable to develop a sub-millimetre fibre optic needle-like Raman probe for minimally invasive deep organ tissue characterization.

Among disclosed technologies over the prior art that may relate instruments used for in vivo measurements of bio-matter include GB201401727D0, which describes a probe for providing medical guidance information comprises a hollow needle probe defining an optical path within the needle probe for a collimated laser beam to illuminate a sample, the optical path further providing a return path for light scattered from the sample due to inelastic scatterings such as Raman scattering light or fluorescence.

However, the technology disclosed in GB201401727D0 may not be suitable for deep tissue Raman measurements as its needle-like probe is described to have a diameter in the millimetre range. Moreover, its probe is shown to have a flat end, indicative of its poor light-focusing capabilities, and furthermore, it may not effectively collect scattered light/fluorescence of an excited deep tissue. Accordingly, it is desirable to have a sub-millimetre fibre optic needle-like Raman probe for minimally invasive deep organ tissue characterization, having superior light-focusing capabilities and light collection capabilities for collecting Raman scattered light or fluorescence from the deep tissue, which is provided by the present invention.

### SUMMARY OF INVENTION

An objective of the invention is to provide a probe for Raman spectroscopy for probing deep tissues and organs, a system that incorporates the probe, and its method of using the system thereof. The deep tissues and organs may be targeted cellular complexes within a bio-matter. To achieve this objective, the probe of the present invention has an optical structure and communication line. More specifically, the optical structure is of small size, and further comprises a lens portion and a spacer portion. The propagation of light between the probe and its environment is manipulated by the optical structure for light to be focused towards the deep tissues or focused towards the communication line of the probe.

Advantageously, the present invention provides a sub-millimetre fibre optic needle-like Raman probe which may be inserted into a fine needle for biopsy of bio-matter, such as organs. The probe of the present invention is also made to be disposable and biocompatible for minimal damage to the bio-matter. The sub-millimetre size of the probe also enables deep penetration within a bio-matter, thereby allowing it to probe deep tissues and organs, such as the brain, spinal cord, liver, lung, lymph nodes, breasts, cardiovascular systems, bone, muscles, joints, etc. The probe also allows for rapid label-free Raman measurements, thereby providing instantaneous diagnosis at a molecular level. Moreover, the probe is also duel-compatible with optical biopsy and fine needle aspiration biopsy, thereby making it compatible with generic methods for targeted tissue sampling, liquid biopsy, in vivo diagnostics, and in vitro diagnostics.

The present invention intends to provide a probe for measuring spectra of a target within a bio-matter, comprising an optical structure having a lens portion, and a communication line conjoined to the optical structure. The optical structure of the probe has its exposed end tapered for forming the lens portion that converges light incoming from the communication line towards the target, and converges scattered light incoming from the target towards the communication line.

Preferably, the optical structure of the probe further comprises a spacer portion that is disposed between the lens portion of the optical structure and the communication line.

Preferably, wherein the spacer portion of the optical structure has a length that is not more than about 40 μm.

Preferably, the lens portion is further rounded with it having either one of a partially-spherical shape, a semi-spherical shape or a hemi-spherical shape.

Preferably, the lens portion of the optical structure has a radius that is not more than about 200 μm.

Preferably, the optical structure is a coreless termination fibre with its exposed end polished to form the lens portion and its other end conjoined with the communication line.

Preferably, the communication line is a multimode optical fibre that has its cladding coated with aluminium.

Preferably, the optical structure and the communication line, in conjunction, has a diameter that is not more than about 500 μm.

The present invention further intends to provide a system for measuring spectra of a target within a bio-matter, comprising a light source for providing an input light, a probe having a portion thereof inserted into the bio-matter and reaching the target, having an optical structure with a lens portion, and a communication line conjoined to the optical structure, for directing the input light from the light source towards the target and receiving scattered light from the target as output light, and a spectrum detector, for receiving the output light from the probe to generate raw spectrum data of the target. The probe has its optical structure have its exposed end tapered for forming the lens portion that converges the input light incoming from the communication line towards the target, and converges scattered light incoming from the target towards the communication line.

Preferably, the optical structure of the probe of the system further comprises a spacer portion that is disposed between the lens portion of the optical structure and the communication line.

Preferably, the system further comprises an optical module as an intermediary for directing input light between the light source and the probe, and for directing output light between the probe and the spectrum detector.

Preferably, the optical module of the system further comprises a plurality of openings for allowing the entry or exit of any one or both the input light or output light, a plurality of lenses for collimating any one or both the input light or output light, a bandpass filter for filtering the input light, a dichroic filter for reflecting the output light; and a longpass filter for filtering the output light.

Preferably, the system comprises a computer for processing the raw spectrum data into clean spectrum data.

Preferably, the computer of the system further operates one or more modules for processing the raw spectrum data into clean spectrum data, which includes an input module, a minimisation module, an optimisation module, a background spectrum estimation module, a comparison module, and an output module.

The present invention further intends to provide a method for measuring spectra of a target within a bio-matter, comprises: providing an input light, by a light source, directing the input light from the light source towards the target, by a probe having an optical structure with a lens portion and a communication line conjoined to the optical structure, receiving scattered light from the target as output light, by the probe, and receiving the output light from the probe to generate raw spectrum data, by a spectrum detector. The probe has its optical structure have its exposed end tapered for forming the lens portion that converges the input light incoming from the communication line towards the target, and converges the scattered light incoming from the target towards the communication line.

Preferably, the method further comprises: directing input light between the light source and the probe, by an optical module, and directing output light between the probe and the spectrum detector, by the optical module.

Preferably, the method further comprises the step of processing the raw spectrum data into clean spectrum data, by a computer.

Preferably, the step of processing the raw spectrum data into clean spectrum data, by a computer, further comprises: receiving the raw spectrum data as an input spectrum data, and an independent measurement data, by an input module of computer, and searching for an estimated interference concentration data and an estimated autofluorescence background data, by a minimisation module of computer, using the input spectrum data and the independent measurement data.

Preferably, the step of processing the raw spectrum data into clean spectrum data, by a computer, further comprises: constructing a total background spectrum data, by a background spectrum estimation module of computer, using the estimated interference concentration data, the estimated autofluorescence background data, and the independent measurement data, and constructing an updated input spectrum data, by an optimisation module of computer, using the raw spectrum data and the total background spectrum data.

Preferably, the step of processing the raw spectrum data into clean spectrum data, by a computer, further comprises: comparing the input spectrum data and the updated input spectrum data, by a comparison module of computer, and constructing the clean spectrum data from the raw spectrum data and the total background spectrum data, by an output module of computer.

One skilled in the art will readily appreciate that the invention is well adapted to carry out the objects and obtain the ends and advantages mentioned, as well as those inherent therein. The embodiments described herein are not intended as limitations on the scope of the present invention.

### BRIEF DESCRIPTION OF THE DRAWINGS

To facilitate an understanding of the invention, there is illustrated in the accompanying drawings the preferred embodiments from an inspection of which when considered in connection with the following description, the invention, its construction and operation and many of its advantages would be readily understood and appreciated.

FIG. 1 is a photograph illustrating a probe of the present invention in a micrometre scale.

FIG. 2 is a ray diagram illustrating the probe probing a bio-matter at a certain depth where a target cellular complex resides.

FIG. 3 is a diagram illustrating a setup of a system that uses the probe of the present invention.

FIG. 4 is a flowchart illustrating a first operational flow that describes the use of system 3 of FIG. 3 for obtaining Raman scattered light from a target 21 of bio-matter 2.

FIG. 5 is a flowchart illustrating a second operational flow that describes the extraction of clean Raman spectrum data of target from its raw Raman spectrum data via structured background subtraction performed by a computer.

FIG. 6 is a graph of Raman collection efficiency against probe depth, in particular, further showing a line plot comparison between a flat probe and the probe of the present invention (labelled tapered probe).

FIG. 7 and FIG. 8 are graphs illustrating the maximum photon count in response to variations in the probe fibre length and the integration time. In particular, FIG. 7 is a graph of maximum photon count against probe fibre length. In particular, FIG. 8 is a graph of maximum photon count against probe integration time.

FIG. 9 illustrates a graph of mean maximum fibre background photon count in Raman spectrum regions against excitation power. In particular, further showing a line plot comparison between photon count in the fingerprint region and the photon count in a high-wavenumber region.

FIG. 10 and FIG. 11 illustrates graphs whereby raw Raman spectrum data of a target 21 collected by the probe 1 is converted into clean Raman spectrum data as per steps illustrated in FIG. 5. In particular, FIG. 10 further shows a line plot comparison between raw Raman spectrum data and background spectrum data. In particular, FIG. 11 illustrates the clean Raman spectrum data, which results from the use of the line plots of FIG. 10.

FIGS. 12 to 14 illustrate Raman spectra in one or more tissue samples. In particular, FIG. 12 illustrates graphs of mean tissue Raman spectra of fat, skin, muscle, cartilage, grey matter, and white matter as collected by the probe of the present invention. FIG. 13 illustrates a graph of signal-to-noise (SNR) ratio of the tissues of FIG. 12 at a wavenumber of 1655 cm⁻¹ with respect to integration time. FIG. 14 illustrates a graph of signal-to-noise ratio of the tissues of FIG. 12 at a wavenumber of 2940 cm⁻¹ with respect to integration time.

FIG. 15 and FIG. 16 illustrates Raman spectra graphs for one or more biofluids that were collected using the probe of the present invention. In particular, FIG. 15 illustrates a mean Raman spectra for urine and blood. FIG. 16 illustrates a SNR of the Raman spectra for urine and blood.

FIGS. 17 to 20 illustrate setups and procedures for collecting Raman spectra from a mice brain model. FIG. 17 illustrates a photograph of the mice brain model. FIG. 18 illustrates of a photograph of the probe of the present invention with the mice brain model.

FIG. 19 illustrates mean tissue Raman spectra of the mice model at various depths across the mice brain model. FIG. 20 illustrates graphs of the protein-to-lipid ratios within various depths across the mice brain model based on the data from FIG. 19.

### DETAILED DESCRIPTION OF THE INVENTION

The present invention relates to a novel sub-millimetre fibre optic Raman probe for probing bio-matter, a system that incorporates the probe, and a method of using the system. The invention may also be presented in a number of different embodiments with common elements. According to the concept of the invention, the tip of the probe, where its lens portion resides, is configured to be tapered for it to have a partially spherical or semi-spherical shape. As such, Raman spectroscopy of a bio-matter is optimised.

It should be noted that in the context of the present invention, the term “bio-matter” preferably relates to matter of biological origin or of synthetic biological origin, which may be living or unliving. It may relate to matter that falls within any one of the multi-cellular level, the cellular level, the sub-cellular level, and pre-cellular level of the biological organisation hierarchy. It may also relate to matter that comprises organic molecules or compounds. It may also relate to matter that are by-products or excretions of biological origin. It may also relate to matter that are syntactically made to be bio-compatible with matter of biological origin. Hence, it is to be understood that the probe, and its system and method thereof, is applicable to any form of bio-matter as described.

The invention will now be described in greater detail, by way of example, with reference to the figures. For ease of reference, common reference numerals or series of numerals will be used throughout the figures when referring to the same or similar features common to the figures.

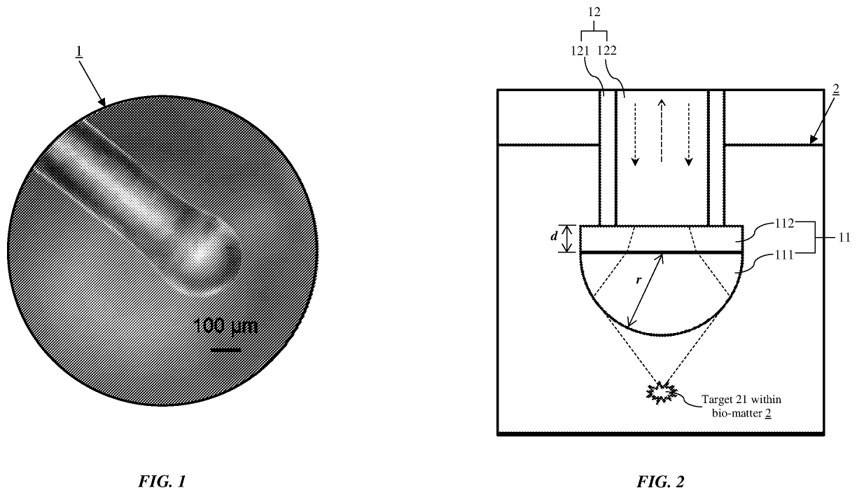

FIG. 1 is a photograph of the tip of a probe 1 of the present invention in a micrometre scale, more specifically, with the probe 1 placed under a microscope with an objective magnification of 10×. This shows the sub-millimetre size of probe 1.

FIG. 2 illustrates the probe 1 probing a bio-matter 2 (e.g. human, animal, plant, etc.) at a certain depth where a target cellular complex 21 (e.g. muscle tissue, fat, blood, urine, artificial cartilage, etc.) resides. In particular, it is shown that probe 1 comprises an optical structure 11 and a communication line 12. The optical structure 11 emits light to induce Raman scattering of the target 21 and subsequently collects its Raman scattered light (which may be synonymous with its fluorescence), while the communication line 12 allows communication between the probe 1 with one or more devices, such as a light source and/or a spectrum detector, to be enabled, for Raman spectroscopy to be performed.

It is to be noted that the bio-matter 2 and the target 21 may have a hierarchical cellular relationship, e.g., bio-matter 2 is a brain organ and target 21 is grey matter tissue. However, this may not necessarily be the case, and bio-matter 2 and the target 21 may be one and the same. While this description shall assume that bio-matter 2 and the target 21 have a hierarchical cellular relationship, this description is to be interpreted to be applicable to cases where bio-matter 2 and the target 21 are one and the same.

Regarding the optical structure 11 of probe 1, it further comprises a lens portion 111 and a spacer portion 112. The optical structure 11 is preferably a coreless termination fibre or a coreless end cap. It may also be any other structure that is able to at least converge light incoming from the communication line 12 towards the bio-matter 2, and converge Raman scattered light incoming from the bio-matter 2 towards the communication line 12. Most preferably, the optical structure 11 is FG250LA from Thorlabs Incorporated, having a diameter of 250 μm and a refractive index of 1.45.

In particular, the lens portion 111 of the optical structure 11 is an exposed end of the optical structure 11. Moreover, the lens portion 111 is tapered and/or rounded, with it preferably being either one a partially-spherical shape, semi-spherical shape or hemi-spherical shape. This is to support its focusing of light. Typically, a coreless termination fibre is an optical terminator that diverges light, however, should it be polished to be tapered and/or rounded, it may subsequently obtain optical properties that are similar to a convex lens in which its focusing capabilities may be attributed thereto. Preferably, the lens portions 111 has a radius r that is substantially not more than 200 μm, but most preferably at about 125 μm. Alternatively, the lens portion 111 may be tapered to have sharp angles similar to a trapezoidal shape, though such a shape may have reduced light focusing capabilities.

In particular, the spacer portion 112 of the optical structure 11 is a body of the optical structure 11 that is conjoined to or fused with the communication line 12. The spacer portion 112 is to provide a gap defined to be between lens portion 111 of the optical structure 11 and a corresponding end of the communication line 12. More specifically, the spacer portion 112 provides a gap d having a length that is substantially not more than 40 μm, but most preferably, 25 μm. The spacer portion 112 is to optimise the propagation of light between the lens portion 111 and the communication line 12 so that light is bent with minimum scattering while travelling therebetween. More specifically, as shown in FIG. 2, it may support in diverging light incoming from the communication line 12 towards the lens portion 111, and in converging Raman scattered light incoming from the lens portion 2 towards the communication line 12. Furthermore, if so desired, the spacer portion 112 may be omitted from the optical structure 11 during its fabrication.

Regarding the communication line 12 of probe 1, it preferably provides light-based communication. It may be any one of a single-mode fibre or a multi-mode fibre. Furthermore, the selections of any one of these fibres is to take into account the wavelengths of the light source and the Raman scattered light of target 21. It is most preferable that communication line 12 is a multi-mode fibre for transmission and receipt of different modes of light between the probe 1 and the one or more devices, such as the light source and/or the spectrum detector. Moreover, regarding the structure of the communication line 12, it has a fibre core 122, and a cladding 121 that is further coated with metallic material such as aluminium or the like. Most preferably, the communication line 12 is AFM200L from Thorlabs Incorporated, being a step index multimode fiber, having a core diameter of 200 μm, a coating diameter of 300 μm, and a numerical aperture (NA) of 0.22.

With this, the fabrication process of probe 1 may be briefly described. In a first step, a strip of coreless termination fibre (for optical structure 11) and a strip of multi-mode fibre coated with aluminium (for communication line 12) are prepared. In a second step, corresponding ends of the coreless termination fibre and the multi-mode fibre are polished. In a third step, the strip of coreless termination fibre is cut for it to be of the intended length of the optical structure 11, this length may preferably include either one or both the radius r of the lens portion 111 and the gap d of the spacer portion 112. In a fourth step, the cut coreless termination fibre has one of its end fused with an end strip of multi-mode fibre, preferably through splicing. A fifth step may be done where the exposed end of the coreless termination fibre is further polished to have a tapered shape so as to form the lens portion 111. With this, the probe 1 with the optical structure 11 and the communication line 12 is fabricated. This ease of fabrication supports its disposability after use.

For probe 1 to be suitably sized for its intended applications, it preferably has an overall outer diameter, inclusive of both the optical structure 11 and the communication line 12, that is not more than 500 μm. It may preferably 100 μm or 300 μm, but may be further reduced to 50 μm. Such a diameter allows the probe 1 to be sheathed within a hyperdermic needle tubing (i.e. a hyperdermic tubing with a sharp end), a fine needle for optical biopsy, or a fine needle for aspiration biopsy, for the optical structure 11 of the probe 1 may reach a target 21 of the bio-matter 2. Furthermore, probe 1 preferably has an overall length, inclusive of both the optical structure 11 and the communication line 12, that is of about 10 cm. Alternatively, the overall length of probe 1 may be between about 6.5 cm to about 20 cm, or at the very least, a length that allows the optical structure 11 to reach the target 21. Under the guidance of the needle tubing, the probe 1 may be allowed to access deep organs, intravascular systems and central nervous systems of a multi-cellular organism.

In regards to the parameters of the probe 1, its optical structure 11 preferably has a refractive index of approximately 1.4536 so as to probe the target 21 that has a refractive index of 1.3. It is noted that the optical structure 11 may not be limited to having a parameter as such, and it may have any other refractive index as long as its refractive index is substantially larger than the refractive index of the target 21.

In regards to the parameters of the probe 1, its optical structure 11 preferably has an effective focal length (EFL) of approximately 224 μm, further having an in-focal spot radius of 127 μm.

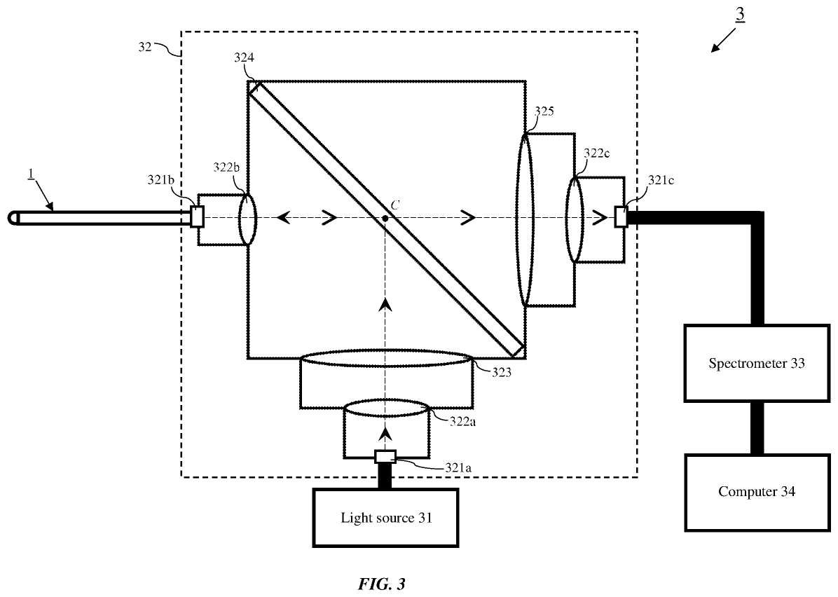

FIG. 3 illustrates a setup of a system 3 that uses the probe 1 of the present invention. As shown, system 3 comprises the probe 1, a light source 31, an optical module 32, a spectrum detector 33, and a computer 34.

Regarding the light source 31 of the system 3, it preferably emits a beam of light towards the optical module 32. In particular, the beam of light is a laser light having a wavelength of between 700 nm to 900 nm, most preferably 785 nm. In particular as well, the light source 31 may allow the power of the beam of light to be adjustable, preferably in a range between 0 mW and 60 mW. However, it is to be noted that the wavelength and power of the beam of light emitted by the light source may be of any value that allows Raman spectroscopy to be performed without damaging the target 21.

Regarding the optical module 32 of the system 3, it is shown to further comprise a plurality of openings 321a, 321b, 321c, a set of lenses 322a, 322b, 322c, a bandpass filter 323, a dichroic filter 324, and a longpass filter 325. The optical module 32 is to act as an intermediary between the light source 31, the probe 1, and the spectrum detector 33. The body of the optical module 32 may be shaped so as to secure all of the aforementioned components therein.

In particular, the plurality of openings 321a, 321b, 321c of the optical module 32 include a first opening 321a, a second opening 321b, and a third opening 321c. Each of them are preferably at right angles with respect to each other about an arbitrary centre point C of the optical module 32. These openings 321a, 321b, 321c allow the entrance or exit of light into or out from the optical module 32. The first opening 321a may be substantially connected to the light source 31 through fibre optics, the second opening 321b may be connected to the probe 1 through a coaxial connection with its communication line 12, and the third opening 321c may be substantially connected to the spectrum detector 33 through fibre optics. The probe 1 may further be sheathed within a needle tubing.

In particular, the set of lenses 322a, 322b, 322c include a first lens 322a, a second lens 322b, and a third lens 322c. Preferably, they are aspheric lenses or any type of light-collimating lenses, for optimising excitation of the light of the light source 31 and Raman couplings. In particular, each of them may be disposed about an opening of the optical module 32. More specifically, the first lens 322a is positioned to be in the vicinity of the first opening 321a, the second lens 322b is positioned to be in the vicinity of the second opening 321b, and the third lens 322c is positioned to be in the vicinity of the third opening 321c.

In particular, the bandpass filter 323 filters light having wavelengths that fail to fall within a specific wavelength range. More specifically, it preferably filters light that has a wavelength dissimilar to the wavelength of the beam of light provided by the light source 31. In particular, it is positioned at the first opening 321a and is in front of the first lens 322a. The bandpass filter 323 is to also attenuate noise that may be present within the beam of light provided by the light source 31.

In particular, the dichroic filter 324 is a dichroic mirror that provides a reflection of light based on their wavelengths. In particular, it is positioned within the optical module 32 at a self-rotated angle of preferably 45° about the centre point C, whereby one side of the dichroic filter 324 substantially faces the first opening 321a and the second opening 321b, and its other side substantially faces the third opening 321c.

In particular, the longpass filter 325 provides filtering of light that is below a certain wavelength. More specifically, it preferably filters wavelengths within the Raman scattered light that may be similar to the wavelength of the light provided by the light source 31. In particular, it is positioned at the third opening 321c and is in front of the third lens 322c. The longpass filter 325 is to also attenuate high-frequency noise that may be present within the Raman scattered light incoming from the target 21 of bio-matter 2.

The spectrum detector 33 is preferably an analytical instrument that performs Raman spectroscopy such as a spectrometer or a spectrograph. In particular, it shall derive parameters of the Raman scattered light of target 21, which may include its wavelength, wavenumber, its signal-to-noise ratio (SNR), and the like, and subsequently record them. Preferably, it is equipped with an array of light sensors, which may be based on charge-coupled device (CCD) technology or active-pixel sensor (APS) technology, for detecting the Raman scattered light. In regards to its hardware and software, it is preferably equipped with a processer running an application software that performs signal processing for deriving and recording a raw Raman spectrum representative of the Raman scattered light of the target 21.

The computer 34 is also preferably an analytical instrument for deriving a clean Raman spectrum from the raw Raman spectrum received from the spectrum detector 33 via structured background subtraction. This task would be further elaborated on in this description. The computer 34 may be interfaced with the spectrum detector via data cables. In regards to its hardware and software, it is preferably equipped with a processer running an application software that performs this task.

Further details on the specific specifications of the light source 31, the optical module 32, and the spectrum detector 33 are as follows.

The light source 31 for generating a 785 nm laser beam is most preferably a laser device such as CleanLaze® laser device series from B&W TEK Incorporated. The light source 31 is also capable of outputting a laser beam having a power ranging between 0 mW to about 600 mW. The power of the light source 31 may be controlled using a variable neutral-density filter, which may be incorporated within the light source 31 or a separate article. The neutral-density filter is most preferably an NDC-50C-2M-B from Thorlabs Incorporated.

The spectrum detector 33 is most preferably an Acton LS-785 f/2 from Princeton Instrument Incorporated. It may further provide high-throughput reflective imaging with a grating density of between 800 gr/mm and 850 gr/mm, but most preferably 830 gr/mm. The spectrum detector 33 is preferably further equipped with a camera or image sensors which is most preferably a PIXIS 400BR-excelon from Princeton Instrument Incorporated. The camera is based on deep-depletion charge-coupled device (DD-CCD) technology, and is further enhanced for use in near-infrared (NIR) imaging, making it compatible for use in Raman spectroscopy.

Moreover, the spectrum detector 33 may have its wavelength and wavenumber axis calibrated using mercury-argon lamps and 4-acetamidophenol for the fingerprint (FP) region and the high-wavenumber (HW) region respectively. Preferably, the system intensity response was calibrated using a standard reference material, with it being NIST 2241 from the National Institute of Standards and Technology.

The optical module 32 most preferably has its bandpass filter 323 rated at a wavelength of 785 nm, most preferably being an LL01-785 from Semrock Incorporated. Its dichroic filter 324 is rated at 785 nm for separation of the light of the light source 31 and the Raman scattered light of the target 21. Its longpasss filter 325 is rated at a wavelength 785 nm.

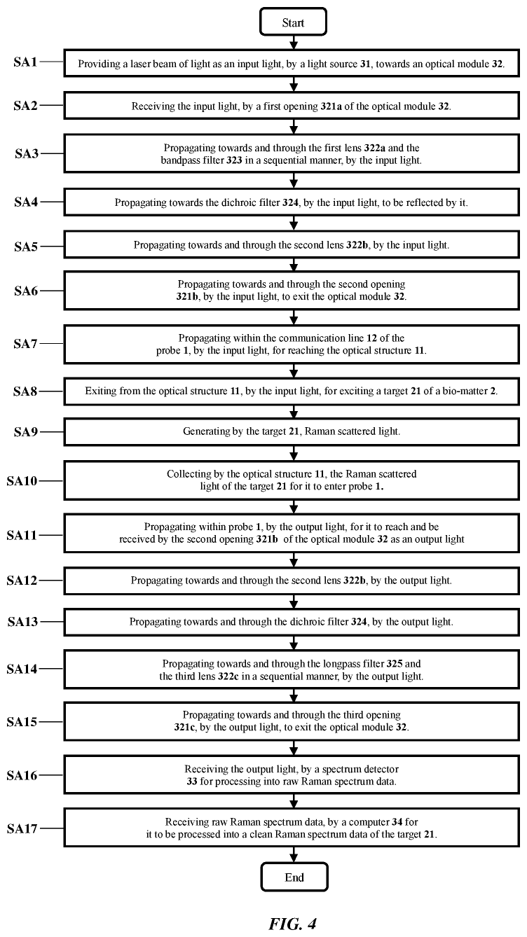

FIG. 4 is a flowchart illustrating a first operational flow that describes the use of system of FIG. 3 for obtaining Raman scattered light from the target 21 of bio-matter 2. It is noted that the steps described in this flowchart are to be interpreted as non-limiting, and minor modifications to the steps (e.g. additions, omissions, or swaps) are permissible by a skilled person without substantial deviation from as described.

It is noted that FIG. 4 inherently assumes that Raman spectroscopy is ready to be performed, whereby the probe 1 has already been inserted into a bio-matter 2 at a certain depth for its optical structure 11 to substantially reach and/or face the target 21.

First, in step SA1, the light source 31 provides a laser beam of light towards the optical module 32. Furthermore, from here on, it is noted that the beam of light emitted by the light source 31 may be referred to as an “input light” as the beam of light is to travel and reach the target 21 of bio-matter 2.

Next, in step SA2, the input light is received by the optical module 32 via its first opening 321a.

Next, in step SA3, the input light propagates towards and through the first lens 322a, and then the bandpass filter 323, in a sequential manner. The input light is collimated by the first lens 322a and then filtered by the bandpass filter 323.

Next, in step SA4, the input light propagates towards the dichroic filter 324 and is reflected by it. More specifically, the dichroic filter 324 reflects the input light towards the second lens 322b.

Next, in step SA5, the input light propagates towards and through the second lens 322b and passes through it to be further collimated.

Next, in step SA6, the input light propagates towards and through the second opening 321b and it exits the optical module 32 therefrom. With this, the input light is now within communication line 12 of the probe 1.

Next, in step SA7, the input light propagates within the communication line 12 to reach the optical structure 11.

Next, in step SA8 the input light exits from the optical structure 11 for it to reach and excite the target 21 of the bio-matter 2.

Next, in step SA9, the target 21, having been stimulated by the input light 21, scatters the input light, thereby generating Raman scattered light.

Next, in step SA10, the Raman scattered light is collected by the optical structure 11 for it to enter the probe 1.

Next, in step SA11, the Raman scattered light propagates back towards the optical module 21 through the communication line 12 of probe 1 to reach and be received by the optical module 32 via its second opening 321b. Furthermore, from here on, it is noted that the Raman scattered light may be referred to as an “output light” as the Raman scattered light is to travel and reach the spectrum detector 33.

Next, in step SA12, the output light passes through the second lens 322b, whereby it is collimated by it.

Next, in step SA13, the output light propagates towards and through the dichroic filter 324 and passes through it. More specifically, the dichroic filter 324 is transparent to the output light.

Next, in step SA14, the output light propagates towards and through the longpass filter 325 and then the third lens 322c, in a sequential manner. The output light is filtered by the longpass filter 325, and then further collimated by the third lens 322c.

Next, in step SA15, the output light propagates towards and through the third opening 321c to exit the optical module 32 therefrom. With this, the output light travels towards the spectrum detector 33.

Next, in step SA16, the output light is received by the spectrum detector 33. Here, the spectrum detector 33 processes the output light into a raw Raman spectrum data.

Finally, in step SA17, the raw Raman spectrum data is received by the computer 34 from the spectrum detector 33 for it to be processed into a clean Raman spectrum data that accurately represents the target 21. Thus ends the description of the first operational flow.

FIG. 5 is a flowchart illustrating a second operational flow that describes the extraction of clean Raman spectrum data of the target 21 from its raw Raman spectrum data via structured background subtraction, which is preferable a task performed by the computer 34. It is noted that the steps described in this flowchart are to be interpreted as non-limiting, and minor modifications to the steps (e.g. additions, omissions, or swaps) are permissible by a skilled person without substantial deviation from as described.

It is further emphasised that the computer 34 that performs the task described in the FIG. 5 is preferably equipped with a processer that operates modules to perform the task. More specifically, the computer 34 may operate a collection of software modules or hardware modules that may correspond to at least one aspect of the task. These modules may include an input module, a minimisation module, a total background spectrum estimator module, an optimisation module, a comparison module, and an output module.

It is also noted that the second operational flow described in the flowchart of FIG. 5 may be an extension of the flowchart of FIG. 4. More specifically, the steps described in the flowchart of FIG. 5 may be regarded as sub-steps of step SA17 in the flowchart of FIG. 4.

It is also noted that the second operational flow of FIG. 5 for extraction of clean Raman spectrum data of the target 21 via structured background subtraction assumes that the raw Raman spectrum data S_{RAW} of the target 21 has 3 parts: (i) fibre Raman and fluorescent background data S_{FIBRE} (i.e. Raman background of the communication line 12) with its interference concentration C_{INT}, (ii) autofluorescence background data S_{AUTO} of the target 21, and (iii) clean Raman spectrum data S_{CLEAN} of the target 21.

It is also noted that the term “data” in the context of the invention refers to a plurality of discrete points in the form of ordered pairs or n-tuples that may be plotted within a coordinate space (e.g., the Cartesian coordinate space, etc.).

First, in step SB1, an input module of computer 34 receives a raw Raman spectrum data S_{RAW} of the target 21 from the spectrum detector 33.

Next, in step SB2, an input module of computer 34 considers the raw Raman spectrum data S_{RAW} as an input Raman spectrum data S_{IN(n)}. Upon reaching this step, n is designated to be=1.

Next, in step SB3, a measured fibre Raman and fluorescent background data S_{FIBRE} is received by the input module of computer 34. In particular, this parameter may be from an independent measurement data that was collected before or after the collection of the raw Raman spectrum data S_{RAW}, and was subsequently uploaded or keyed into the computer 34.

Next, in step SB4, S_{IN(n)} and S_{FIBRE} are provided to a minimisation module of computer 34 for it to search for an estimated interference concentration data C_{INT(est._n)} and an estimated autofluorescence background data S_{AUTO(est._n)} of target 21 for a current n^{th} iteration, whereby n=1, 2, 3 . . . . Preferably, the minimisation module performs this search through the use of Algo. 1.

$$arg {min}_{( {C}_{INT ( est\_n )} , {S}_{AUTO ( est . \_ n )} )} [ {S}_{IN ( n )} - ( {C}_{INT ( est . \_ n )} ) ( {S}_{FIBRE} ) - {S}_{AUTO ( est . \_ n )} \qquad ( Algo . 1 )$$

The objective function (in square brackets) of Algo. 1 is calculated iteratively for minimum data values of C_{INT(est._n)} and S_{AUTO(est._n)} of the current n^{th} iteration to be found based on the known data values of S_{IN(n)} and S_{FIBRE} as constraints. Preferably, upon a certain number of iterations or upon reaching a certain preset value, the minimum data values of C_{INT(est._n)} and S_{AUTO(est._n)} of the current n^{th} iteration are found.

Next, in step SB5, C_{INT(est._n)} (found in step SB4), S_{AUTO(est._n)} (found in step SB4), and S_{FIBRE} (from step SB3) are provided to the total background spectrum estimation module of computer 34. The total background spectrum estimation module constructs an estimated total background spectrum data S_{BG(est._n)} by means of Eq. 1 and curve fitting. Curve fitting was done for fitting the discrete data from Eq. 1 into a continuous function.

$${S}_{BG ( est . \_ n )} = ( {C}_{INT ( est . \_ n )} ) ( {S}_{FIBRE} ) + {S}_{AUTO ( est {.}_{n} )} \qquad ( Eq . 1 )$$

Next, in step SB6, the optimisation module of computer 34 compares the input Raman spectrum data S_{IN(n)} and the estimated total background spectrum data S_{BG(est._n)} of the current n^{th} iteration to construct an updated input Raman spectrum data S_{IN(n_u)}. Preferably, the data points along the updated input Raman spectrum data S_{IN(n_u)} are constructed according to the conditions below, with S_{IN(n)} and S_{BG(est._n)} having equal or approximately equal abscissas.
1. For ordinate data points of S_{IN(n)} that are larger than the ordinate data points of S_{BG(est._n)}, the ordinate data points of S_{BG(est._n)} are used in the construction of S_{IN(n_u)}.
2. For ordinate data points of S_{IN(n)} that are smaller or equal to the ordinate data points of S_{BG(est._n)}, the ordinate data points of S_{IN(n)} are used in the construction of S_{IN(n_u)}.

With this, the updated input Raman spectrum data S_{IN(n_u)} constructed in step SB6 has minimised background error.

Next, as per step SB7, the comparison module of computer 34 compares the input Raman spectrum data S_{IN(n)} of the current n^{th} iteration and the updated input Raman spectrum data S_{IN(n_u)}. For the sake of clarity, S_{IN(n)} may be also be referred to as a prior input Raman spectrum data S_{IN(n_p)}. More specifically, the computer 34 will determine their absolute percentage difference.

The next step SB8, is a decision step, whereby it will be determined whether or not the absolute percentage difference between S_{IN(n_p)} and S_{IN(n_u)} less than 1%. Should this be the case, step SB8 proceeds to step SB9. Else, step SB8 proceeds to step SB11.

In Step SB9, since it was determined that the absolute percentage difference between the data of S_{IN(n_p)} and S_{IN(n_u)} is less than 1%, the output module of computer 34 will then nominate data. In particular, it will consider the most recent n^{th} iteration of the prior input Raman spectrum data S_{IN(n_p_r)}, and the most recent n^{th} iteration the estimated total background spectrum data S_{BG(est._n_r)} as nominated data.

Following Step SB9 is SB10. Here, output module of the computer 34 constructs the clean Raman spectrum data S_{CLEAN} of the target 21 using the nominated data (S_{IN(n_p_r)} and S_{BG(est._n_r)}) by means of Eq. 2 and curve fitting. Curve fitting done for fitting the discrete data from Eq. 2 into a continuous function. This marks the end of the second operational flow and the clean Raman spectrum data S_{CLEAN} of the target 21 is obtained.

$${S}_{CLEAN} = {S}_{IN ( n\_p \_r )} - {S}_{BG ( est . \_n \_r )} \qquad ( Eq . 2 )$$

In step SB11, since it was determined that the absolute percentage difference between S_{IN(n_p)} and S_{IN(n_u)} is not less than 1%, S_{IN(n_u)} is then reserved for use for a succeeding iteration as S_{IN(n)} whereby it is provided to the minimisation module. With this, step SB11 returns to step SB4 and interactively loops therefrom.

Thus ends the description of the second operational flow.

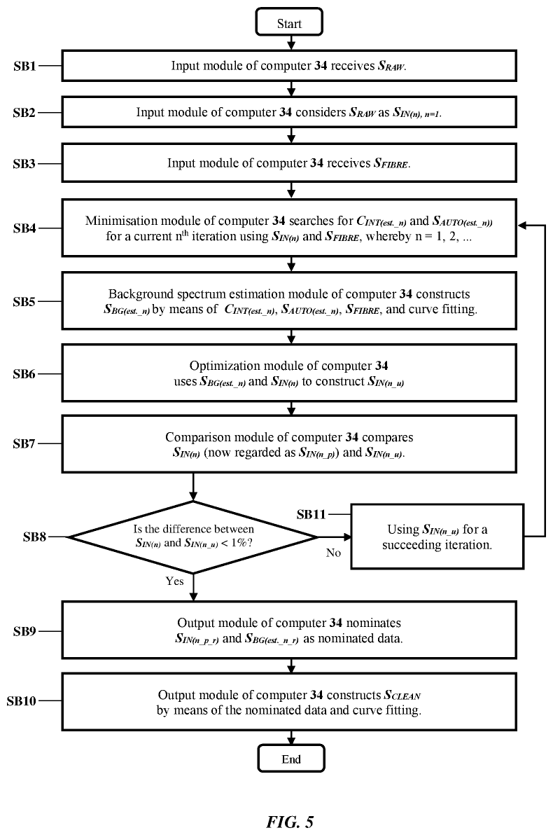

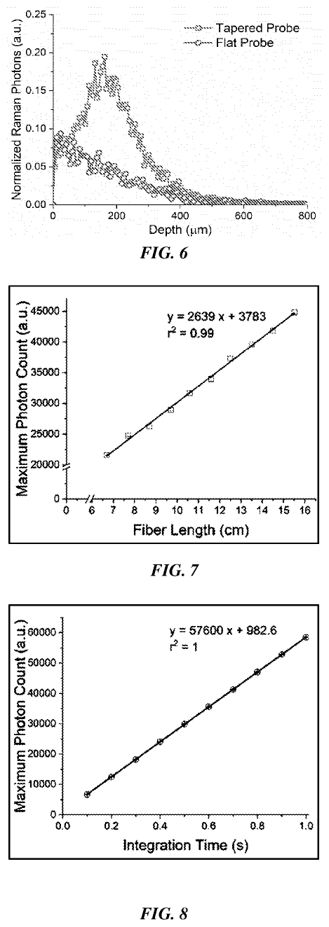

FIGS. 6 to 20 relate to experimental data collected using the probe 1 of the present invention. More specifically, experimental data collected using the system 3 having the probe 1, through the use of the steps described in the flowcharts of FIG. 4 and FIG. 5, which intend to illustrate the performance of probe 1, its system 3 and the method of using the system 3.

FIG. 6 is a graph of normalised Raman photon collection efficiency against depth of the target 21 within bio-matter 2. In particular, it further shows a line plot comparison between a flat probe and the probe 1 of the present invention (labelled tapered probe), which are obtained via simulations. As shown, in the depths ranging from 0 to about 800 μm, the probe 1 of the present invention provides Raman collection that is improved by approximately 3.03 times compared to the flat probe. This indicates the improved excitation focusing and Raman signal collection capability of the probe 1 due to the sub-millimetre size and the tapered shape of its optical structure 11.

FIG. 7 and FIG. 8 are graphs illustrate the mean maximum photon count in response to variations in the fibre length (i.e. length of communication line 12 of probe 1) and the integration time of the spectrum detector 33 (more specifically, the integration time of the photodetectors of the spectrum detector 33). In particular, FIG. 7 is a graph of maximum photon count against fibre length the probe 1. In particular, FIG. 8 is a graph of maximum photon count of the probe 1 against the integration time of the spectrum detector 33.

FIG. 7 illustrates mean maximum photon counts (e.g., 800 cm⁻¹) with a standard deviation (SD) of +1 using the probe 1 having its fibre length varied from about 6.5 cm to about 16 cm. The probe 1 is within the system 3 where its light source 31 provides an input light having an excitation power of about 30 mW, and its spectrum detector 33 has an integration time of about 0.5 s. Based on of this graph, the probe 1 may have a fibre length of up to about 20 cm under aforementioned specifications of the system 3 before system 3 reaches its saturation point (i.e., 65535 photon counts per pixel).

FIG. 8 illustrates mean maximum photon counts (e.g., 800 cm⁻¹) with SD of +1 using the probe 1 in the system 3 where the integration time of the spectrum detector 33 of system 3 varied from about 0.1 s to 1 s. The system 3 also has its light source 31 provides an input light having an excitation power of about 30 mW.

As shown in both FIG. 7 and FIG. 8, the performance of the probe 1 of the present invention indicates a linear relationship (r²≈1) of its mean maximum photon counts with respect to the fibre length of the probe 1, and the integration time of the spectrum detector 33. This substantiates the robustness of the structured background subtraction algorithms developed for clean Raman spectrum retrieval of the target 21.

FIG. 9 illustrates a graph of mean maximum photon count in Raman spectrum regions against excitation power of the light source 31. In particular, it further shows a line plot comparison between photon count in the spectrum of the fingerprint (FP) region and the photon count in the spectrum of the high-wavenumber (HW) spectrum region.

In particular, the graph of FIG. 9 is plotted by measuring the spectrum in the FP region and the HW region using input light of different excitation powers. More specifically, it illustrates mean maximum photon counts with SD of +1 obtained using the probe 1 at wavenumbers of 800 cm⁻¹ and 2800 cm⁻¹, whereby the probe 1 has a fibre length of about 10 cm, and its system 3 has its light source 31 provides an input light having an excitation power ranging from 0 mW to about 50 mW and its spectrum detector 33 has an integration time of about 1 s. As shown, a linear response is exhibited when power of the input light is less than 35 mW, thereby indicating that the saturation of spectrum detector 33 may be prevented by controlling the excitation power of the input light to be less than about 35 mW, thereby providing the possibility of recovering the Raman signal in both FP and HW regions.

FIG. 10 and FIG. 11 illustrates graphs whereby raw Raman spectrum data of a target 21 collected by the probe 1 is converted into clean Raman spectrum data as per steps illustrated in FIG. 5. In particular, FIG. 10 further shows a line plot comparison between raw Raman spectrum data and background spectrum data. In particular, FIG. 11 illustrates the clean Raman spectrum data, which results from the use of data present in FIG. 10.

In particular, FIG. 10 and FIG. 11 show an example of subtraction of fluorescent background and fibre background from using the raw Raman spectrum data. The raw Raman spectrum data is collected from porcine fat using probe 1, whereby its system 3 has a light source 31 that provides an input light having an excitation power 30 mW and a spectrum detector 33 having an integration time of 0.7 s.

Raw Raman spectrum data ranging from 800 cm⁻¹ to 3300 cm⁻¹ was collected from the porcine fat and processed by computer 34 as per the flowchart of FIG. 5. Preferably, for reducing processing load, only raw Raman spectrum in the FP region (ranging from 800 cm⁻¹ to 1800 cm⁻¹) and the HW region (ranging from 2800 cm⁻¹ to 3300 cm⁻¹) are processed by computer 34, with the silent region (ranging from 1800 cm⁻¹ to 2800 cm⁻¹) that lacks Raman contribution being selectively ignored. The resulting clean Raman spectrum data for these regions are fitted curves plotted using polynomial functions, with a fifth-order polynomial fitting used for the spectrum in the FP region, and a second-order polynomial fitting used for the spectrum in the HW region. It is noted that the spectral intensity in the HW region has been amplified with a factor of around 10 for better visualization.

By accurately estimating the fluorescent background and fibre background as shown in FIG. 10, the clean Raman spectrum data of the porcine fat could be successfully recovered in both the FP region and HW region as shown in FIG. 11. This indicates the feasibility of obtaining Raman measurements using the probe 1 as it is an improvement that is about 1.78 times better than using a flat fibreoptic Raman probe. This further indicates that the probe 1 of the present invention has an improved depth selection capability.

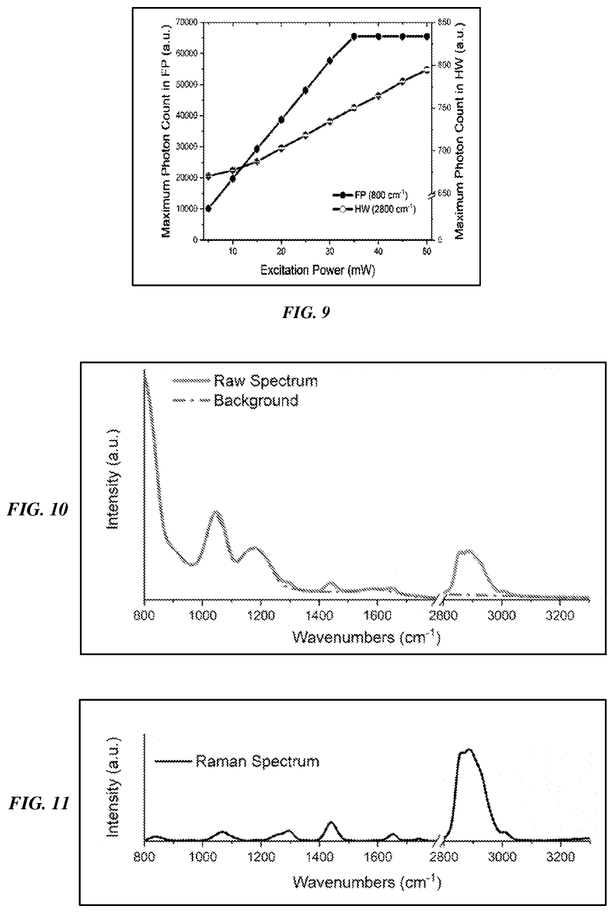

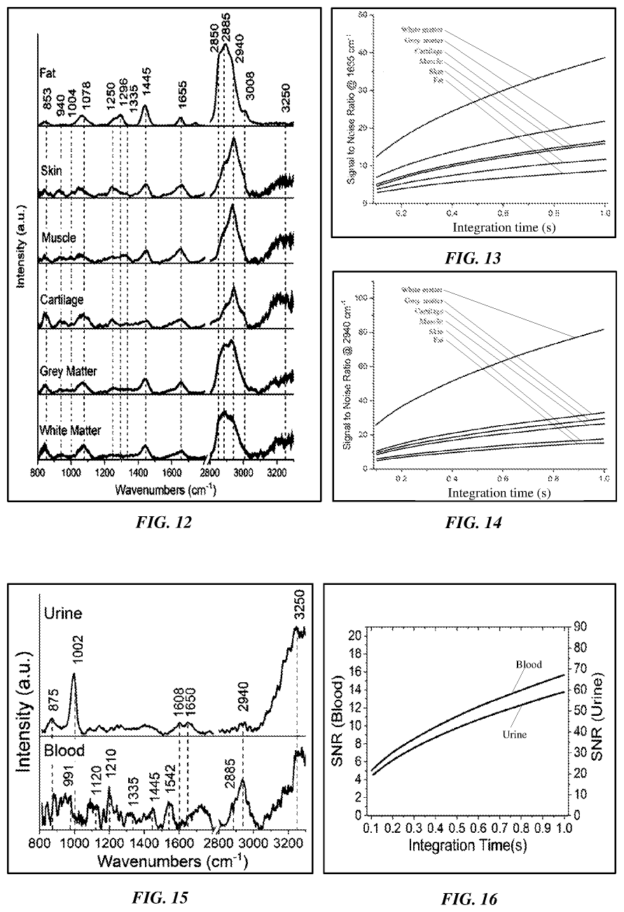

FIGS. 12 to 14 illustrate Raman spectra collected from one or more tissue samples using the probe 1.

In particular, FIG. 12 illustrates that the probe 1 was validated using various tissue samples as target 21, which include porcine skin, porcine muscle, and porcine fat, chicken cartilage, murine grey matter, and murine white matter, under a system 3 that has its light source 31 provides an input light having an excitation power of 30 mW and its spectrum detector 33 has having an integration time ranging from 0.1 s to 1 s.

FIG. 12 illustrates the mean normalized Raman spectra of the tissue samples with SD of +1, in a system 3 where its spectrum detector 33 has an integration time of 0.5 s. As shown, distinct Raman peaks are observed in both the FP region and the HW region.

More specifically, in the FP region of FIG. 12, there are Raman peaks approximately observed at wavenumbers that include 853 cm⁻¹ (indicative of v(C—C) proteins), 1004 cm⁻¹ (indicative of v(C—C) ring breathing of phenylalanine), 1078 cm⁻¹ (indicative of v(C—C) of lipids), 1250 cm⁻¹ (indicative of Amide III), 1296 cm⁻¹ (indicative of CH₂ deformation), 1335 cm⁻¹ (indicative of CH₃CH₂ twisting of proteins and nucleic acids), 1445 cm⁻¹ (indicative of CH₂ deformation of proteins and lipids, and 1655 cm⁻¹ (indicative of Amide I and C═C of lipids), etc.

More specifically, in the HP region of FIG. 12, there are Raman peaks approximately observed at wavenumbers that include 2850 cm⁻¹ (indicative of CH₂ symmetric stretching of lipids), 2885 cm⁻¹ (indicative of CH₂ asymmetric stretching of lipids), 2940 cm⁻¹ (indicative of C—H vibration in lipids and proteins), and 3250 cm⁻¹ (indicative of OH stretching), etc.

With this, FIG. 12 has shown that the probe 1 allows for the confirmation of unique bio-molecular compounds (e.g., lipids, nucleic acid, and proteins, etc.) that are present in the different tissue samples. This is because of the unique Raman features and peaks for different samples (e.g., Raman peak intensities and peak width, etc.) are observed at wavenumbers ranging from 1200 cm⁻¹ to 1500 cm⁻¹ in the FP region, and wavenumbers ranging from 2800 cm⁻¹ to 3000 cm⁻¹ in the HW region.

FIG. 13 and FIG. 14 illustrate the change in SNR with respect to the integration time for different tissue samples. FIG. 13 illustrates a graph of SNR of the tissues of FIG. 12 of a Raman peak in the FP region having wavenumber of 1655 cm⁻¹ with respect to integration time. FIG. 14 illustrates a graph of SNR of the tissues of FIG. 12 of a Raman peak in the HW region having wavenumber of 2940 cm⁻¹ with respect to integration time. As shown in both figures, the SNRs in both the FP and HW regions show an increasing trend with respect to the square root of the integration time. Also, at an integration time of 0.5 s, the SNR of the representative Raman peaks in both the FP and HW regions are at least higher than 5, thereby indicating the effective Raman signal acquisition capability of the designed probe 1 Raman spectra collection in multiple organ sites.

FIG. 15 and FIG. 16 illustrate Raman spectrum graphs for one or more biofluids that were collected using the probe 1 of the present invention. In particular, FIG. 15 illustrates a mean Raman spectra for urine and blood. FIG. 16 illustrates a SNR of the Raman spectra for urine and blood.

FIG. 15 and FIG. 16 demonstrate the ability of the probe 1 for rapid acquisition of Raman spectrum data from biofluids that are mice blood and mice urine under a system 3 that has its light source 31 provides an input light having an excitation power of 30 mW and its spectrum detector 33 having integration times ranging from 0.1 s to 1 s.

FIG. 15 illustrates the mean Raman spectra of both mice blood and mice urine with a SD of +1, whereby the system 3 has its spectrum detector 33 has with an integration time of 0.5 s. Here, prominent biofluidal Raman peaks located within the FP region are approximately observed at wavenumbers that include 875 cm⁻¹, 991 cm⁻¹ (indicative of RBC, phenylalanine and NADH), 1002 cm⁻¹ (indicative of N—C—N stretching of urea), 1120 cm⁻¹ (indicative of carotene), 1210 cm⁻¹ (indicative of RBC), 1335 cm⁻¹, 1445 cm⁻¹, 1542 cm⁻¹ (indicative of RBC and Amide II), 1608 cm⁻¹ (indicative of urea), etc. Here, prominent biofluidal Raman peaks located within the HW region are approximately observed at wavenumbers that include 2885 cm⁻¹, 2940 cm⁻¹, 3250 cm⁻¹, etc.

As shown in FIG. 15, the unique Raman features of urine attributed to urea (e.g., Raman peaks at wavenumbers of 1002 cm⁻¹, 1608 cm⁻¹, etc.) and the Raman features of blood originating from haemoglobin and RBCs (e.g., Raman peaks at wavenumbers of 991 cm⁻¹, 1210 cm⁻¹, and 1542 cm⁻¹) are observed, affirming the excellent performance of the probe 1 in identifying different bio-molecular structures of different biofluids.

FIG. 16 shows the SNR of the Raman spectra of mice urine and mice blood with respect to the integration time of the spectrum detector 33 that varies from 0.1 s to 1 s. More specifically, the SNR of the Raman peak of mice urine is derived from the wavenumber of 1003 cm⁻¹, and the SNR of the Raman peak of mice blood is derived from the wavenumber of 1542 cm⁻¹. Here, it is further observed that the SNR of the Raman peaks for both biofluid samples are higher than 10 at an integration time of ˜0.5 s. Moreover, the SNR of both Raman spectra increases with respect to the square root of integration time. This demonstrates the robustness of the probe 1 for rapid and quantitative Raman measurements of biofluids as it provides a high SNR response.

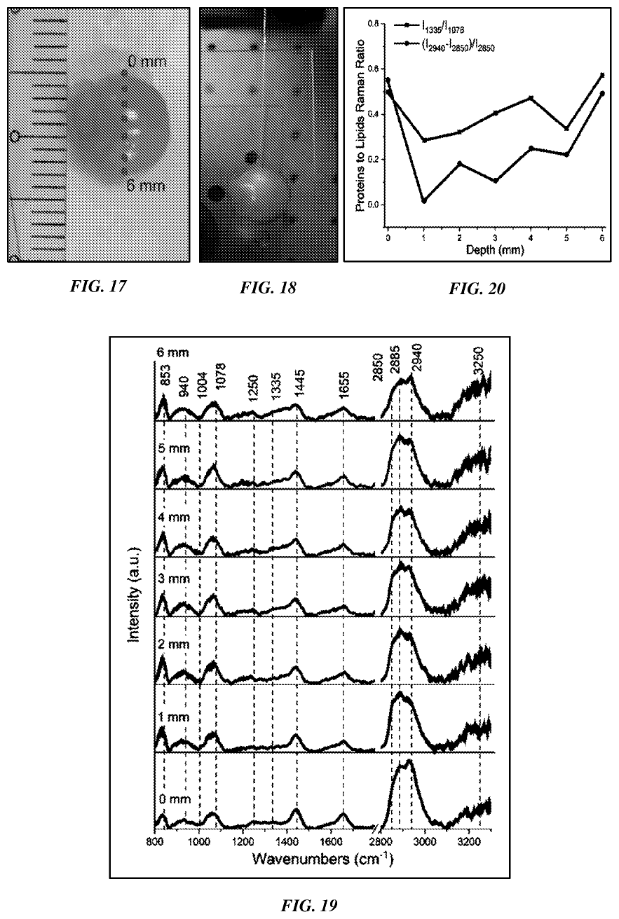

FIGS. 17 to 20 illustrate the performance assessment of probe 1 using a mice brain model. These figures illustrate setups and procedures for collecting Raman spectra from the mice brain model. FIG. 17 illustrates a photograph of the mice brain model. FIG. 18 illustrates of a photograph of the probe of the present invention with the mice brain model. FIG. 19 illustrates mean Raman spectrum of the mice model at various depths across the mice brain model. FIG. 20 illustrates graphs of the protein-to-lipid ratios within various depths across the mice brain model based on the data from FIG. 19.

As shown in FIG. 17 and FIG. 18, the probe 1 is inserted into the mice brain model at 7 different depths (i.e. 0 mm, 1 mm, 2 mm, 3 mm, 4 mm, 5 mm, and 6 mm). The direction of insertion is from the model's left parietal cortex to the model's right parietal cortex. At each depth, ten Raman spectra of the FP and HW regions were acquired using the probe 1 under a system 3 that has its light source 31 provides an input light having an excitation power of 30 mW and its spectrum detector 33 having an integration time of 0.5 s.

FIG. 19 shows the mean Raman spectra of the mice brain model at different depths with a standard deviation (SD) of +1. Signature Raman peaks of brain tissue mentioned previously were observed at all depths (i.e. approximately at wavenumbers of 853 cm⁻¹, 1004 cm⁻¹, 1078 cm⁻¹, 1250 cm⁻¹, 1335 cm⁻¹, 1445 cm⁻¹, 1655 cm⁻¹ in the FP region; and approximately at wavenumbers of 2850 cm⁻¹, 2885 cm⁻¹, 2940 cm⁻¹, and 3250 cm⁻¹ in the HW region).

Moreover, from FIG. 19, Raman spectral features variations, which include changes in its peak intensity and peak widths, could be observed at different depths of the mice brain model. More specifically, peak intensity changes were observed approximately at wavenumbers of 853 cm⁻¹, 1004 cm⁻¹, 1078 cm⁻¹, 1445 cm⁻¹, 1655 cm⁻¹, 2885 cm⁻¹, 2940 cm⁻¹ and 3250 cm⁻¹. Peak width changes were observed approximately at wavenumbers of 1078 cm⁻¹, 1445 cm⁻¹, and 1655 cm⁻¹. These Raman spectral features variations reflect bio-molecular differences at different brain anatomical locations of the mice brain model. From this, the proteins-to-lipids Raman ratios at different depths were investigated.

FIG. 20 shows plots of mean protein-to-lipid Raman ratio with a standard deviation of +1, which are plotted using representative Raman peaks related to proteins and lipids in both the FP and HW regions. Representative Raman peaks in the FP regions include the wavenumbers of 1078 cm⁻¹ (indicative of v(C—C) of lipids) and 1335 cm⁻¹ (indicative of CH₃CH₂ twisting of proteins and nucleic acids). Representative Raman peaks in the HW regions include the wavenumbers of 2850 cm⁻¹ (indicative of CH₂ symmetric stretching of lipids) and 2940 cm⁻¹ (indicative of C—H vibration in lipids and proteins). The protein-to-lipid Raman ratio for the FP region is estimated using the formula I₁₃₃₅/I₁₀₇₈. Whereas, the protein-to-lipid Raman ratio for the HW region is estimated using the formula ((I₂₉₄₀−I₂₈₅₀))/I₂₈₅₀.

As shown in FIG. 20, the mean protein-to-lipid Raman ratio for both FP and HW regions decreases at the depths of 0 mm to about 1 mm. This is because probe 1 was inserted from the outer layer grey matter of the cerebral cortex, which has more protein and nucleic acid-rich neuron cells compared to the central core of white matter located at about 1 mm deep that has various lipids-rich glial support cells.

As shown in FIG. 20, the mean protein-to-lipid Raman ratio for both FP and HW regions starts to increase at the depths of about 2 mm to 4 mm. This is because the probe 1 was inserted into the thalamus region, which is rich in proteins and functional neurons.

As shown in FIG. 20, the mean protein-to-lipid Raman ratio for both FP and HW regions drops when a depth of about 5 mm is reached, but increases when the depth of about 6 mm is reached. This is because the probe 1 passes through the white matter regions (located at about 5 mm depth) and grey matter regions (located at about 6 mm depth) of the right side of the cerebral cortex.

The bio-molecular information observed in the Raman ratios was obtained at different depths in FIG. 20 confirms the depth-resolved Raman signal collection ability of the probe 1. The correlation coefficient r of the proteins-to-lipids Raman ratios between the two plots was calculated to be around 0.82, proving the high Raman signal consistency between the Raman peaks in FP and HW regions.

With this, the details pertaining to the novel sub-millimetre fibre optic Raman probe 1 for probing bio-matter, the system 3 that incorporates the probe, and a method of using the system 3 have been sufficiently elucidated. Its potential to be widely used in biomedical applications that involve Raman spectroscopy has also been elucidated.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

1.-20. (canceled)

21. A probe for measuring Raman spectra of a target within a bio-matter, comprising
an optical structure having a lens portion; and
a communication line conjoined to the optical structure;
wherein the optical structure has an exposed end tapered for forming the lens portion and a spacer portion, so that the lens portion and the spacer portion converge light incoming from the communication line towards the target, and converge scattered light incoming from the target towards the communication line.

22. The probe according to claim 21, wherein the spacer portion of the optical structure has a length that is not more than about 40 μm.

23. The probe according to claim 21, wherein the lens portion is further rounded to either one of a partially-spherical shape, a semi-spherical shape or a hemi-spherical shape.

24. The probe according to claim 21, wherein the lens portion of the optical structure has a radius that is not more than about 200 μm.

25. The probe according to claim 21, wherein the optical structure is a coreless termination fibre with the exposed end polished to form the lens portion and the spacer portion.

26. The probe according to claim 21, wherein the communication line is a multimode optical fibre that has its cladding coated with aluminium.

27. The probe according to claim 21, wherein the optical structure and the communication line, in conjunction, has a diameter that is not more than about 300 μm.

28. A system for measuring Raman spectra of a target within a bio-matter, comprising:
a light source for providing an input light;
a probe having a portion thereof inserted into the bio-matter and reaching a target located in the bio-matter, the probe having an optical structure with a lens portion and a spacer portion, and a communication line conjoined to the optical structure, for directing the input light from the light source towards the target and receiving output light, scattered light from the target; and
a spectrum detector, for receiving the output light from the probe to generate raw spectrum data of the target;
wherein the probe and the optical structure have an exposed end tapered to form the lens portion and the spacer portion that converge the input light incoming from the communication line towards the target, and converge scattered light incoming from the target towards the communication line.

29. The system according to claim 28, further comprising an optical module as an intermediary for directing input light between the light source and the probe, and for directing output light between the probe and the spectrum detector.

30. The system according to claim 29, wherein the optical module further comprises:
a plurality of openings for allowing the entry or exit of any one or both the input light or output light;
a plurality of lenses for collimating any one or both the input light or output light;
a bandpass filter for filtering the input light;
a dichroic filter for reflecting the input light; and
a longpass filter for filtering the output light.

31. The system according to claim 28, further comprising a computer for processing the raw spectrum data into clean spectrum data.

32. The system according to claim 31, wherein the computer further operates one or more modules for processing the raw spectrum data into clean spectrum data, which includes an input module, a minimisation module, an optimisation module, a background spectrum estimation module, a comparison module, and an output module.

33. A method for measuring Raman spectra of a target within a bio-matter, comprising the steps of:
providing an input light, by a light source;
directing the input light from the light source towards a target, by a probe having:
an optical structure with a lens portion and a spacer portion; and
a communication line conjoined to the optical structure;
receiving scattered light from the target as output light, by the probe; and
receiving the output light from the probe to generate raw spectrum data, by a spectrum detector;
wherein the probe and the optical structure have an exposed end tapered for forming the lens portion and the spacer portion that converge the input light incoming from the communication line towards the target, and converge the scattered light incoming from the target towards the communication line.

34. The method according to claim 33, further comprising the steps of:
directing input light between the light source and the probe, by an optical module; and
directing output light between the probe and the spectrum detector, by the optical module.

35. The method according to claim 34, further comprising the step of:
processing the raw spectrum data into clean spectrum data, by a computer.

36. The method according to claim 35, wherein the step of processing the raw spectrum data into clean spectrum data, by a computer, further comprising the steps of:
receiving the raw spectrum data as an input spectrum data, and an independent measurement data, by an input module of the computer; and
searching for an estimated interference concentration data and an estimated autofluorescence background data, by a minimisation module of the computer, using the input spectrum data and the independent measurement data.

37. The method according to claim 36, further comprising the steps of:
constructing a total background spectrum data, by a background spectrum estimation module of the computer, using the estimated interference concentration data, the estimated autofluorescence background data, and the independent measurement data; and
constructing an updated input spectrum data, by an optimisation module of the computer, using the raw spectrum data and the total background spectrum data.

38. The method according to claim 37, further comprising the steps of:
comparing the input spectrum data and the updated input spectrum data, by a comparison module of the computer; and
constructing the clean spectrum data from the raw spectrum data and the total background spectrum data, by an output module of the computer.
